(19) United States
(12) Patent Application Publication
(10) Pub. No.: US 2010/0210719 A1
(43) Pub. Date: Aug. 19, 2010

(54) O-DESMETHYLVENLAFAXINE

(75) Inventors: Surya Narayana Devarakonda, Hyderabad (IN); Sesha Reddy Yarraguntla, Hyderabad (IN); Venu Nalivela, Warangal (IN); Ram Thaimattam, Hyderabad (IN); Subbareddy Peddireddy, Cuddapah (IN); Balaji Raghupati, Hyderabad (IN); Mohammed Azeezulla Baig, Vijayawada (IN); Srinivas Reddy Gade, Hyderabad (IN); Srinivas Reddy Mallepalli, Hyderabad (IN); Vijaywardhan Chitta, Ranga Reddy (IN); Satyanarayana Bollikonda, Hyderabad (IN); Saravanan Mohanarangam, Valavanur (IN); Rajasekhar Kadaboina, Hyderabad (IN)

Correspondence Address: DR. REDDY''S LABORATORIES, INC., 200 SOMERSET CORPORATE BLVD, SEVENTH FLOOR, BRIDGEWATER, NJ, 08807-2862, US

(73) Assignees: DR. REDDY'S LABORATORIES LTD., Hyderabad 500016 Andhra Pradesh, AP (IN); DR. REDDY'S LABORATORIES, INC., Bridgewater, NJ (US)

(21) Appl. No.: 12/668,685

(22) PCT Filed: Jul. 10, 2008
(86) PCT No.: PCT/US08/69653

§ 371 Date: Jan. 12, 2010

Related U.S. Application Data
(60) Provisional application No. 61/037,920, filed on Mar. 19, 2008.
(60) Provisional application No. 61/060,192, filed on Jun. 10, 2008.
(60) Provisional application No. 61/073,070, filed on Jun. 17, 2008.

(30) Foreign Application Priority Data
Jul. 12, 2007 (IN) 1502/CHE/2007
Oct. 16, 2007 (IN) 2341/CHE/2007
Nov. 19, 2007 (IN) 2680/CHE/2007

(51) Int. Cl. A61K 31/277; A61K 31/137; C07C 255/37; C07C 215/42; C07C 209/48; C07C 209/84; C07C 213/00; A61P 25/24

(52) U.S. Cl. 514/520; 514/653; 558/410; 564/355; 564/357; 564/437

(57) ABSTRACT

Processes for preparing desvenlafaxine and stable amorphous O-desmethylvenlafaxine succinate solid dispersions with one or more pharmaceutically acceptable carriers.

### INTRODUCTION

The present invention relates to an improved process for the preparation of O-desmethylvenlafaxine, its intermediates and its pharmaceutically acceptable salts. It also relates to amorphous and crystalline solid forms of O-desmethylvenlafaxine succinate, methods for their preparation and their pharmaceutical compositions.

O-desmethylvenlafaxine or desvenlafaxine are adopted names for the drug compound having a chemical name 1-[2-dimethylamine(4-hydroxyphenyl)ethyl]cyclohexanol, and represented by structural Formula I.

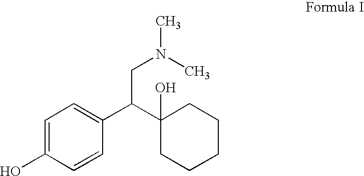

O-desmethylvenlafaxine is prescribed for treating major depressive disorders. O-desmethylvenlafaxine, the major metabolite of venlafaxine, selectively blocks the reuptake of serotonin and norepinephrine and is currently marketed in the U.S. under the trademark PRISTIQ® in the form of sustained-release tablets containing 50 mg and 100 mg of the drug, for oral administration.

Various processes using a variety of intermediates, reagents, solvents and conditions have been reported in the literature for the preparation of O-desmethylvenlafaxine. However, they all have some disadvantages associated with their use.

U.S. Pat. No. 4,535,186 discloses O-desmethylvenlafaxine and its pharmaceutically acceptable salts. Further, it discloses a process for preparing a O-desmethylvenlafaxine fumarate salt. It also discloses a process for the preparation of venlafaxine, which involves the catalytic hydrogenation of phenylacetonitrile derivatives using a rhodium catalyst. It discloses a process for the preparation of O-desmethylvenlafaxine that involves use of a benzyl blocking group on the 4-hydroxy group of the phenyl ring, which leads to relatively low yields.

U.S. Pat. No. 6,350,912 discloses a process for the preparation of venlafaxine in a single vessel. In this patent, a cyano derivative is reduced in the presence of Raney nickel in a mixture of ammonia and ethanol. However, the yield appears to be relatively low.

U.S. Pat. No. 7,026,513 discloses the hydrogenation of 1-[cyano(4-methoxyphenyl)methyl]cyclohexanol to form 1-[2-amino-1-(4-methoxyphenyl)ethyl]cyclohexanol using Nickel Corm III catalyst. However, this process leads to formation of high levels of impurities.

International Application Publication No. WO 2000/76955 describes a process for preparing (R)-desmethylvenlafaxine, which involves the use of sodium hydride as a base to form a sodium salt of ethanediol, which subsequently would be treated with venlafaxine.

U.S. Pat. No. 6,689,912 describes a process for preparation of O-desmethylvenlafaxine, where the formation of dodecanethiolate is followed by treatment with venlafaxine in the presence of polyethylene glycol.

U.S. Pat. No. 7,026,508 describes a process for preparation of O-desmethylvenlafaxine, which involves demethylating venlafaxine or a salt thereof with an alkali metal salt of a trialkylborohydride.

International Application Publication No. WO 00/59851 describes a process for preparation of O-desmethylvenlafaxine, which involves contacting venlafaxine with lithium diphenylphosphide for a time and at a temperature sufficient to form O-desmethylvenlafaxine.

International Application Publication No. WO 2007/071404 describes a process for preparation of O-desmethylvenlafaxine, which comprises combining metal sulfide, venlafaxine, and optionally selenium in a solvent and heating it sufficiently to obtain O-desmethylvenlafaxine.

International Application Publication No. WO 2007/120923 describes a process for preparation of O-desmethylvenlafaxine, which comprises combining venlafaxine, an organic solvent and a reagent selected from the group consisting of thiophenol, sodium sulfide and a C₁-C₈ alkyl thiolate, heating the mixture and recovering O-desmethylvenlafaxine.

The above processes involve use of hazardous, toxic, costly and highly difficult-to-use reagents, which is not desirable on a production scale. Also the yield and purity appear to be relatively low.

U.S. Pat. No. 6,673,838 discloses O-desmethylvenlafaxine succinate and four crystalline forms of O-desmethylvenlafaxine succinate, designated as Form I, Form II, Form III, and Form IV, and an amorphous form of O-desmethylvenlafaxine succinate.

U.S. Pat. No. 6,673,838 discloses an amorphous form of desvenlafaxine succinate. The patent further discloses that the glass transition (T_{g}) onset for the amorphous form occurs at 18° C. According to differential scanning calorimetry, the amorphous form shows a major endotherm at about 120° C. (FIG. 6 of the patent). Without being bound by any theory, it is possible that the amorphous form was converted into a crystalline form before reaching 120° C., since amorphous forms typically do not exhibit endotherms, while crystalline forms do. This phenomenon clearly indicates that the amorphous form that is disclosed in U.S. Pat. No. 6,673,838 is highly unstable and is not desirable for use in pharmaceutical formulations.

International Application Publication No. WO 2008/017886 discloses O-desmethylvenlafaxine succinate hydrate.

### SUMMARY OF THE INVENTION

An aspect of the present invention provides an improved process for the preparation of highly pure 1-[2-amino-1-(4-methoxyphenyl)ethyl]cyclohexanol or a pharmaceutically acceptable salt thereof, which process is simple, cost-effective and also easy to operate on a production scale.

An aspect of the present invention provides an improved process for the preparation of a substantially pure O-desmethylvenlafaxine of Formula I or a pharmaceutically acceptable salt thereof, which process is simple, cost-effective, does not involve toxic and hazardous reagents, and also easy to operate on a production scale.

An aspect of the present invention provides a stable amorphous solid dispersion of O-desmethylvenlafaxine succinate and processes for its preparation.

An aspect of the present invention provides new crystalline forms of O-desmethylvenlafaxine succinate and processes for their preparation.

An aspect of the present invention provides an improved process for the preparation of the compound of Formula IV,

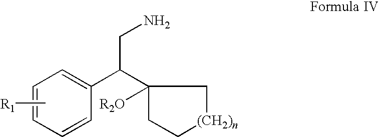

by hydrogenation of phenylacetonitrile of Formula V,

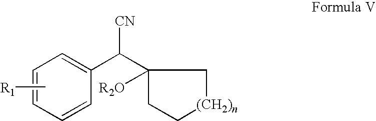

wherein: R₁ is H, —OH, amino, alkylamino, alkylamido, halo, unsubstituted or substituted alkyl or alkoxy; R₂ is hydrogen or a hydroxy protecting group; and n is 1, 2 or 3; in the presence of an activated nickel catalyst. The compound of Formula IV may be further converted to its pharmaceutically acceptable salts.

An aspect of the present invention provides improved processes for the synthesis of O-desmethylvenlafaxine of Formula I, an embodiment comprising:

(1) reacting dodecanethiol with a suitable base in presence of a suitable solvent to afford the metal salt of dodecanethiol of Formula III; and

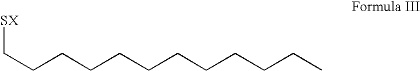

(2) reacting venlafaxine hydrochloride of Formula II with the metal salt of dodecanethiol of Formula III obtained in (1) in the presence of a suitable organic solvent under suitable reaction conditions to afford the desired compound of Formula I, and optionally converting the compound of Formula I into a pharmaceutically acceptable salt.

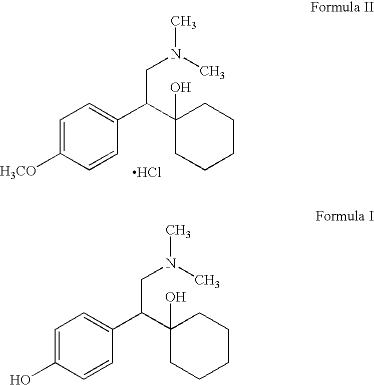

An aspect of the present invention provides purification processes for the compound of Formula I.

An aspect of the present invention provides purification processes for the compound of Formula I, an embodiment comprising recrystallization of the O-desmethylvenlafaxine from a suitable organic solvent to afford the desired substantially pure compound of Formula I.

An aspect of the present invention provides stable amorphous solid dispersions of O-desmethylvenlafaxine succinate, in combination with a pharmaceutically acceptable carrier.

An aspect of the present invention provides processes for the preparation of stable amorphous solid dispersions of O-desmethylvenlafaxine succinate in combination with a pharmaceutically acceptable carrier, an embodiment comprising removing the solvent from a solution comprising O-desmethylvenlafaxine succinate and one or more pharmaceutically acceptable carriers.

An aspect of the present invention provides processes for preparing O-desmethylvenlafaxine succinate, an embodiment comprising reacting O-desmethylvenlafaxine with succinic acid in presence of a suitable solvent. Examples of suitable solvents include but are not limited to water, alcohols, ethers, hydrocarbon solvents, esters, nitriles, and mixtures thereof.

An aspect of the present invention provides a new crystalline form of O-desmethylvenlafaxine succinate, hereinafter referred to as “Form V.”

An aspect of the present invention provides processes for the preparation of crystalline Form V of O-desmethylvenlafaxine succinate, an embodiment comprising crystallizing or slurrying O-desmethylvenlafaxine succinate in a solvent or a mixture of solvents for a suitable period of time sufficient to provide Form V. Examples of suitable solvents include but are not limited to dimethylformamide (DMF), N,N-dimethylacetamide (DMA), and mixtures thereof.

An aspect of the present invention provides a new crystalline form of O-desmethylvenlafaxine succinate, hereinafter referred as “Form VI.”

An aspect of the present invention provides processes for the preparation of crystalline Form VI of O-desmethylvenlafaxine succinate, an embodiment comprising crystallizing or slurrying O-desmethylvenlafaxine succinate in a solvent or a mixture of solvents, for a period of time sufficient to provide Form VI of O-desmethylvenlafaxine succinate. Examples of suitable solvents include but are not limited to dimethylsulfoxide (DMSO), dimethylformamide (DMF), methyl isobutyl ketone (MIBK), ethyl methyl ketone, and mixtures thereof.

An aspect of the present invention provides pharmaceutical compositions comprising a therapeutically effective amount of at least one solid form of O-desmethylvenlafaxine succinate described herein and at least one pharmaceutically acceptable excipient.

An aspect of the present invention provides pharmaceutical compositions comprising a therapeutically effective amount of a solid dispersion of O-desmethylvenlafaxine succinate along with a pharmaceutically acceptable carrier described herein, and at least one pharmaceutically acceptable excipient.

### BRIEF DESCRIPTION OF THE DRAWINGS

FIG. 1 is an X-ray powder diffraction (XRPD) pattern for crystalline Form V of O-desmethylvenlafaxine succinate.

FIG. 2 is an XRPD pattern for crystalline Form VI of O-desmethylvenlafaxine succinate.

FIG. 3 is an XRPD pattern of amorphous O-desmethylvenlafaxine succinate solid dispersion, in combination with a pharmaceutically acceptable carrier.

FIG. 4 is a differential scanning calorimetry (DSC) curve of amorphous O-desmethylvenlafaxine succinate form, in combination with povidone.

FIG. 5 is an XRPD pattern of amorphous O-desmethylvenlafaxine succinate solid dispersion in combination with polyethylene glycol 6000.

### DETAILED DESCRIPTION

Purity percentages are expressed herein as weight percentages. All X-ray analytical information was generated using copper Kα radiation.

An aspect of the present invention relates to an improved process for the preparation of the compound of Formula IV,

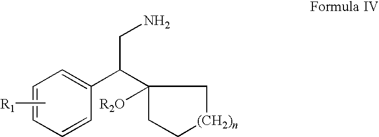

by hydrogenation of a phenylacetonitrile of Formula V:

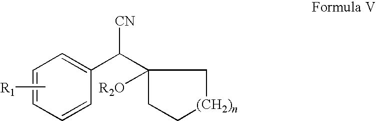

wherein: R₁ is H, OH, amino, alkylamino, alkylamido, halo, or unsubstituted or substituted alkyl or alkoxy; R₂ is hydrogen or a hydroxy protecting group; and n is 1, 2 or 3; in the presence of an activated nickel catalyst. The compound of Formula (IV) may be further converted into any of its pharmaceutically acceptable salts.

In an embodiment, there is provided a process for the preparation of the compound of Formula VI,

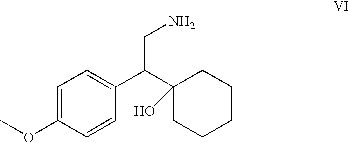

which is an intermediate for the preparation of venlafaxine, by hydrogenation of the compound of Formula VII,

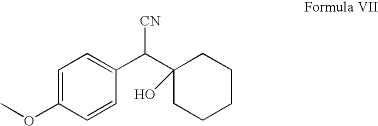

using an activated nickel catalyst, and optionally the compound of Formula VI may be further converted to an acid addition salt such as an acetic acid salt or hydrochloride salt.

The compound of Formula VI or its salt may be further converted to venlafaxine or an acid salt thereof, such as the hydrochloride salt of Formula II.

An embodiment of the synthetic pathway may be illustrated as follows:

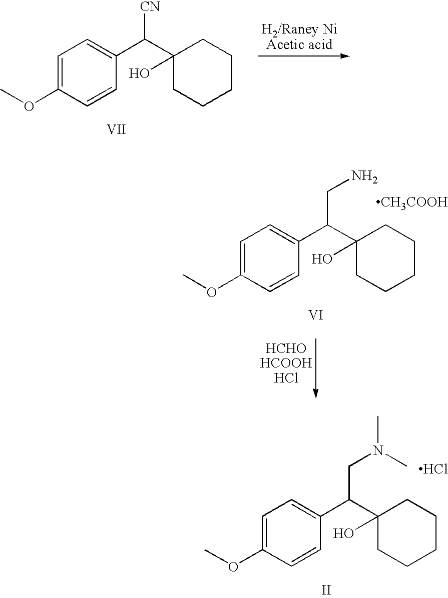

An aspect of the present invention provides processes for the preparation of a compound of Formula VI, an embodiment comprising reducing a compound of Formula VII using an activated nickel alloy catalyst in an organic acid, in an autoclave at a hydrogen pressure of 5-20 Kg/cm² and temperatures in the range of about 30-75° C., until the reduction is substantially complete.

The catalyst may be used in weight proportions of about 100 to about 5 wt. % of the compound of Formula III. In an embodiment, the catalyst concentration is about 15% w/w of the compound of Formula III.

The hydrogenation may be carried out in an organic acid such as formic acid, acetic acid, propionic acid, and the like. After the completion of the reaction, the catalyst may be removed using various techniques such as filtration. The reaction mass may be optionally treated with a base such as ammonia to isolate the product as free base. The product may be extracted into a suitable solvents such as halogenated hydrocarbon solvents, ester solvents, aromatic hydrocarbon solvents, ethers, and the like.

The useful halogenated hydrocarbon solvents include but are not limited to dichloromethane and chloroform. Useful ester solvents include ethyl acetate, propyl acetate and t-butyl acetate. Examples of aromatic hydrocarbon solvents that can be used include toluene and xylenes.

The obtained compound of Formula VI may be further converted to venlafaxine hydrochloride of Formula II, e.g., by the process disclosed in U.S. Patent Application Publication No. 2005/0033088, published on Feb. 10, 2005; which is incorporated herein by this reference in its entirety, or it may also be prepared by any processes known in the art.

Venlafaxine hydrochloride obtained from the above process may be used as a starting material for the preparation of O-desmethylvenlafaxine succinate.

An aspect of the present invention provides improved processes for the preparation of O-desmethylvenlafaxine of Formula I in high yield and purity.

An aspect of the present invention provides improved processes for the preparation of O-desmethylvenlafaxine of Formula I, an embodiment comprising:

(1) reacting dodecanethiol with a suitable base in presence of a suitable solvent to afford a metal salt of dodecanethiol of Formula III; and

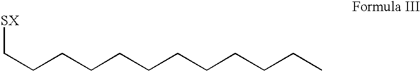

(2) reacting venlafaxine hydrochloride of Formula II with a metal salt of dodecanethiol of Formula III in the presence of a suitable organic solvent under suitable reaction conditions to afford the desired compound of Formula I.

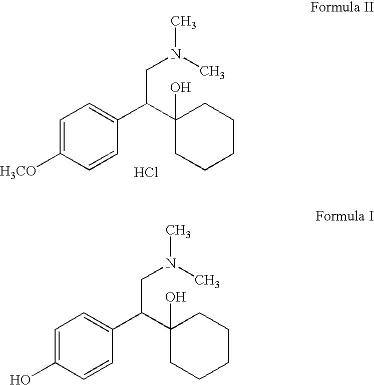

Step (1) involves a reaction of dodecanethiol with a suitable base.

Suitable bases in step (1) that may be used include but are not limited to: inorganic bases such as sodium hydroxide, potassium hydroxide and the like; carbonates of alkali metals such as sodium carbonate, potassium carbonate and the like; bicarbonates of alkali metals such as sodium bicarbonate and potassium bicarbonate. These bases may be used in the form of solids or in the form of aqueous or alcoholic mixtures. Suitably, the molar ratios of the base used in the reaction may range from about 2 to about 5, or about 3, equivalents, per equivalent of dodecanethiol of Formula III.

Suitable solvents for use in step (1) include, but are not limited to, water-immiscible solvents such as hydrocarbon solvents including but not limited to toluene, xylene, n-hexane, n-heptane, cyclohexane, and the like, and mixtures thereof.

Suitable temperatures for conducting the reaction range from about 10° C. to about 150° C. or from about 25° C. to about 40° C., and the suitable reaction times range from about 30 minutes to about 10 hours, or longer. In an embodiment, the reaction time is about 3 hours.

Step (2) involves a reaction of venlafaxine hydrochloride with a salt of dodecanethiol.

Suitable organic solvents, which may be used in step (2) include but are not limited to aprotic polar solvents such as N,N-dimethylformamide (DMF), dimethylsulfoxide (DMSO), N,N-dimethylacetamide (DMA), N-methylpyrrolidone (NMP), hexamethylphosphoramide (HMPA), methyl cellosolve, and the like, and mixtures thereof. Suitable temperatures for conducting the reaction in step (2) range from about 50° C. to about 250° C., or about 100° C. to about 200° C., or about 150° C. to about 175° C., or about the reflux temperature of the solvent used. The suitable times for completion of the reaction depend on the temperature and other conditions, and range from about 30 minutes to about 4 hours, or longer, or can be about 1 hour.

After completion of the reaction, the reaction mass is decomposed by addition of water and the pH of the reaction solution is adjusted to basic values. The formed solid can be recovered by conventional methods including decantation, centrifugation, gravity filtration, vacuum filtration or other techniques known in the art for the recovery of solids. The obtained solid may optionally be further purified by crystallizing from a suitable solvent. The resulting O-desmethylvenlafaxine base may be optionally converted to a salt and the resulting substantially pure salt may be optionally converted to substantially pure O-desmethylvenlafaxine base.

An aspect of the present invention provides processes for the re-crystallization of the O-desmethylvenlafaxine from a suitable organic solvent to afford the desired substantially pure O-desmethylvenlafaxine base.

Recrystallization involves providing a solution of O-desmethylvenlafaxine in a suitable solvent and then crystallizing the solid from the solution. A solution of O-desmethylvenlafaxine may be obtained by dissolving O-desmethylvenlafaxine in a suitable solvent or it may be obtained from a reaction mixture containing the compound. Suitable solvents include but are not limited to: C₁-C₅ ketones, such as acetone, methyl ethyl ketone, butanone and the like; alcohols such as ethanol, methanol, and isopropanol; ethers such as tetrahydrofuran and 1,4-dioxane; esters such as ethyl acetate, propyl acetate, t-butyl acetate and the like; water; and mixtures thereof in various proportions without limitation.

Suitable temperatures for forming a solution range from about 25° C. to about 75° C., or about the reflux temperature of the solvent used. The concentration of the O-desmethylvenlafaxine in the solvent may range from about 5% to about 90%, or more. When higher solute concentrations are desired, the solution may be prepared at an elevated temperature. Any temperature is acceptable for the dissolution as long as a clear solution of the O-desmethylvenlafaxine is obtained and it is not detrimental to the drug substance chemically or physically.

The solution obtained may be optionally treated with activated charcoal, followed by filtration through paper, cloth, or a membrane, or a medium such as a flux-calcined diatomaceous earth (Hyflow) bed, to remove the carbon. Crystal formation from the solution may be promoted by techniques such as cooling, seeding, adding an anti-solvent, and the like. Anti-solvents that may be used include but not limited to aromatic hydrocarbons such as toluene, xylenes and the like, ethers such as diethyl ether, diisopropyl ether and the like, and aliphatic hydrocarbons such as hexanes, n-heptane, cyclohexane, and the like. The solid can then be isolated by centrifugation, filtration, etc., and further dried. Drying may be suitably carried out using a tray dryer, vacuum oven, air oven, fluidized bed drier, spin flash dryer, flash dryer and the like. The drying may be carried out at temperatures from about 25° C. to about 75° C., with or without vacuum and in the presence or absence of an inert atmosphere such as nitrogen, argon, neon, or helium. The drying may be carried out for any desired time periods to achieve the desired product purity, and the times may range from about 1 to about 15 hours, or longer, to obtain a desired residual solvent content.

The processes of the present invention result in high yields of O-desmethylvenlafaxine of Formula I, substantially free from process-related impurities. Frequently, the O-desmethylvenlafaxine that is recrystallized using the process of the present invention is of high purity, such as at least about 99 wt % and the level of impurities may be less than about 1 wt %, or about 0.5 wt %, or about 0.1 wt %, as determined by high performance liquid chromatography (HPLC).

An aspect of the present invention provides pharmaceutical compositions containing a therapeutically effective amount of pure O-desmethylvenlafaxine or a pharmaceutically acceptable salt thereof, containing less than about 0.1% of any individual impurity, together with one or more pharmaceutically acceptable excipients.

An aspect of the present invention provides processes for preparation of O-desmethylvenlafaxine succinate. A reaction between O-desmethylvenlafaxine and succinic acid in the presence of a suitable solvent produces O-desmethylvenlafaxine succinate. Examples of suitable solvents include alcohols, ethers, hydrocarbons, esters, nitriles, and mixtures thereof or their combinations with water in various proportions. O-desmethylvenlafaxine and succinic acid are mixed, such as at about a 1:1 molar ratio, with a sufficient amount of the solvent to provide a solution of O-desmethylvenlafaxine succinate at or below the reflux temperature of the solvent.

Solvents that may be used for dissolution include but are not limited to: alcohols such as methanol, ethanol and isopropyl alcohol; ethers such as 1,4-dioxane, diethyl ether, tetrahydrofuran, diisopropyl ether, and methyl tertiary-butyl ether; hydrocarbons such as toluene, xylene, n-hexane, n-heptane and cyclohexane; esters such as ethyl acetate, n-propyl acetate, n-butyl acetate and tertiary-butyl acetate; nitriles such as acetonitrile and propionitrile; halogenated hydrocarbons such as dichloromethane, ethylene dichloride and chloroform; and mixtures thereof or their combinations with water in various proportions.

An aspect of the present invention provides stable amorphous solid dispersions of O-desmethylvenlafaxine succinate and with a pharmaceutically acceptable carrier.

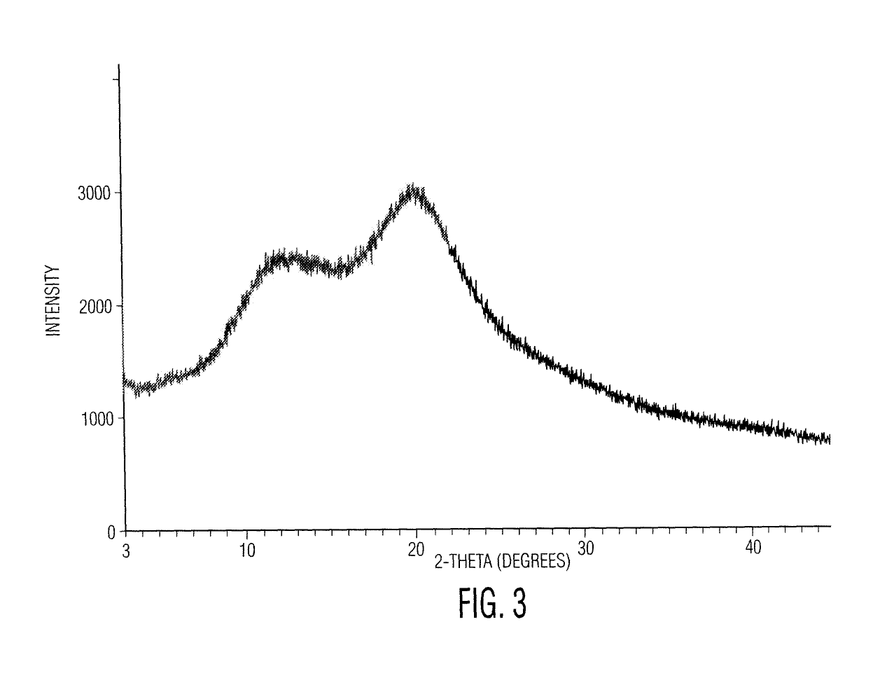

FIG. 3 is an XRPD pattern of an amorphous O-desmethylvenlafaxine succinate solid dispersion in combination with a pharmaceutically acceptable carrier.

An aspect of the present invention provides processes for preparation of a stable solid dispersion of O-desmethylvenlafaxine succinate together with a pharmaceutically acceptable carrier, an embodiment comprising:

a) providing a solution or an admixture of O-desmethylvenlafaxine succinate and one or more pharmaceutically acceptable carriers in a solvent;

b) isolating a solid dispersion from the solution;

c) optionally drying the solid dispersion comprising O-desmethylvenlafaxine succinate and a pharmaceutically acceptable carrier.

Suitable solvents that may be used for providing a solution of O-desmethylvenlafaxine succinate together with one or more pharmaceutically acceptable carriers include but are not limited to, polar and non-polar solvents, and mixtures thereof. Non-limiting examples of suitable solvents include, but are not limited to: polar solvents such as water, methanol, ethanol, n-propanol, isopropanol, n-butanol, isobutanol, t-butanol, 1,4-dioxane, tetrahydrofuran, acetone, acetonitrile, dimethylsulfoxide (DMSO), N-methylpyrolidone (NMP), N,N-dimethylformamide (DMF) and N,N-dimethylacetamide (DMA); non-polar solvents such as n-hexane, benzene, toluene, diethyl ether, chloroform, ethyl acetate, dichloromethane; and mixtures thereof.

The suitable pharmaceutically acceptable carriers which may be used in combination with any form of O-desmethylvenlafaxine succinate include but are not limited to: hydrophilic carriers such as polymers of N-vinylpyrrolidone commonly known as polyvinylpyrrolidine, “PVP,” or “povidone”, gums, cellulose derivatives such as microcrystalline celluloses, ethyl cellulose, hydroxypropyl methylcellulose (HPMC or hypromellose) and hypromellose phthalate, cyclodextrins, gelatins, sugars, polyhydric alcohols, polyethylene glycols (PEG), polyethylene oxides, polyoxyalkylene derivatives, methacrylic acid copolymers, polyvinyl alcohols, and propylene glycol derivatives.

Useful pyrrolidones include, but are not limited to homopolymers or copolymers of N-vinylpyrrolidone. Such polymers can form complexes with a variety of compounds. The water-soluble forms of N-vinylpyrrolidone are available in a variety of viscosity and molecular weight grades such as but not limited to PVP K-12, PVP K-15, PVP K-17, PVP K-25, PVP K-30, PVP K-90, PVP K-120 and crospovidone.

Polyethylene glycols, condensation polymers of ethylene oxide and water, are commercially available from various manufacturers in average molecular weights ranging from about 300 to about 10,000,000 Daltons. Some of the grades that are useful in the present invention include, but are not limited to, PEG 1500, PEG 4000, PEG 6000, PEG 8000, etc.

Any pharmaceutical carrier will be acceptable as long as it allows the formation of the stable amorphous solid dispersion of O-desmethylvenlafaxine succinate as described herein, is compatible with the O-desmethylvenlafaxine succinate, and is acceptable for human pharmaceutical use. The choice of carrier is within the scope of understanding of a person skilled in the art and is not limited by the list of carriers above.

The pharmaceutically acceptable carriers that are used for the preparation of the solid dispersions of the present invention may optionally be pretreated with reagents such as sodium metabisulfite, sodium sulfite, butylated hydroxytoluene, trialkyl amine, aldehydes, alkali or alkaline earth metal hydroxides like sodium hydroxide, potassium hydroxide, dimethylsulfoxide, and the like, in order to remove any contaminants that may cause undesired impurity formation during the preparation of the solid dispersion, which in turn may result in a solid dispersion contaminated with undesired impurities for a pharmaceutical product.

The dissolution temperatures for the O-desmethylvenlafaxine succinate, optionally along with one or more pharmaceutically acceptable carriers, may range from about 0° C. to about 130° C., or the reflux temperature of the solvent used. Any other temperatures may also be acceptable, provided a clear solution of the concerned materials is obtained in the solvents chosen, and the starting materials are not degraded. It will be understood that the temperatures required will also be determined by the processing conditions for the recovery of the final product, such as the temperature of drying, the boiling point of the solvent, the homogeneity of the solution required after mixing solvents, the viscosity of the solution, the stability of the O-desmethylvenlafaxine succinate and the pharmaceutically acceptable carrier. Such variations are all included herein without any limitation.

The solvent may be removed from the solution by techniques such as distillation under vacuum. The solvent may be distilled under reduced pressure maintained at about 1 to 100 mbar, or about 10 to 30 mbar. The distillation may be conducted at a temperature from about 30 to about 125° C., to dryness.

The solvent(s) may be also removed from the solution by techniques known in the art including but not limited to: distillation, evaporation, oven drying, tray drying, rotational drying (such as with the Buchi Rotavapor), spray drying, freeze-drying, fluid bed drying, flash drying, spin flash drying and thin-film drying.

The solid dispersions of O-desmethylvenlafaxine succinate with one or more pharmaceutically acceptable carriers are stable during storage. This property is important and advantageous for the desired use of O-desmethylvenlafaxine succinate in pharmaceutical product formulations.

Individual particles of the original components are not distinguishable in the solid dispersions, using techniques such as optical microscopy. While the invention is not to be bound to any particular theory, the solid dispersions in some instances can be considered to be dispersions at a molecular level, or solid solutions.

The solid dispersions of O-desmethylvenlafaxine succinate in combination with one or more pharmaceutically acceptable carriers of the present invention are stable in the amorphous state, as indicated by the glass transition temperatures observed with differential scanning calorimetry.

The processes described herein may include drying of the product with or without vacuum and in the presence or absence of an inert atmosphere. Other conventional drying methods may also be used.

O-desmethylvenlafaxine succinate used as a starting material may be of any polymorphic form. O-desmethylvenlafaxine or its salt or its precursor intermediate in any polymorphic form may also be used as a starting material for the preparation of crystalline and amorphous O-desmethylvenlafaxine succinate by following the processes described above.

Any form of O-desmethylvenlafaxine such as anhydrous crystalline, amorphous, crystalline hydrate, or mixtures of amorphous and crystalline forms of O-desmethylvenlafaxine in any proportions obtained by any method, will be acceptable for forming a solution.

The stable amorphous solid dispersions of O-desmethylvenlafaxine succinate of the present invention have commercially acceptable pharmacokinetic characteristics, solubility, flow properties, stability, and the like. The products may optionally be milled to get the desired particle size distributions. Milling or micronization may be performed prior to drying, or after the completion of drying of the products. The milling operation reduces the size of particles and increases surface area of particles by colliding particles with each other at high velocities.

An aspect of the present invention provides a new crystalline form of O-desmethylvenlafaxine succinate, named “Form V.”

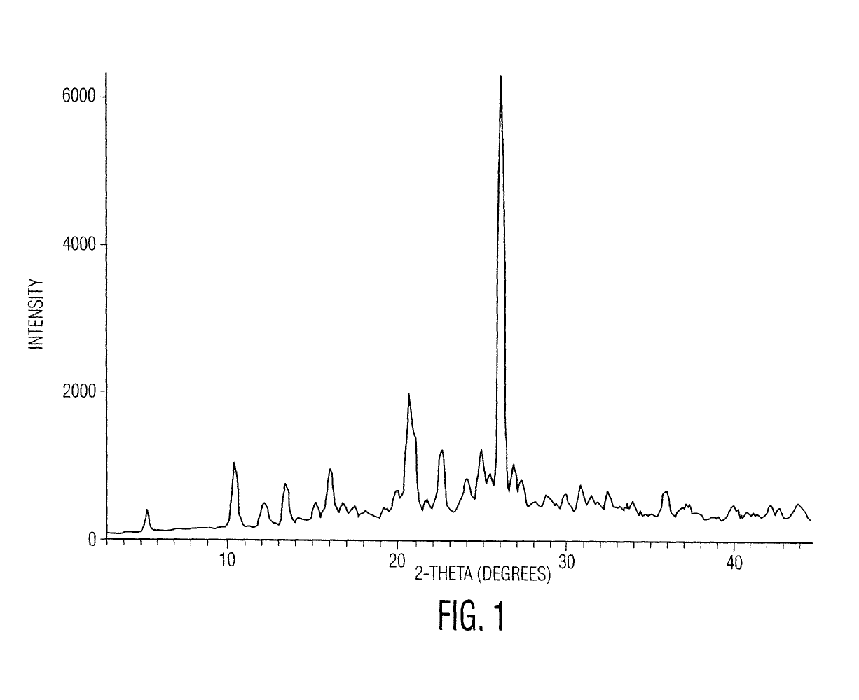

Table 1 contains representative XRPD pattern peak values for crystalline Form V of O-desmethylvenlafaxine succinate. An example of an XRPD pattern for crystalline Form V of O-desmethylvenlafaxine succinate is shown in FIG. 1.

TABLE 1

| 2-Theta | | Intensity |
|---|---|---|
| (degrees) | d (Å) | (%) |
| 10.4 | 8.5 | 13.4 |
| 10.6 | 8.3 | 8.6 |
| 15.9 | 5.5 | 11.1 |
| 16.2 | 5.4 | 11.7 |
| 20.4 | 4.3 | 16.2 |
| 20.6 | 4.3 | 28.5 |
| 21.0 | 4.2 | 15.2 |
| 22.4 | 3.9 | 16.01 |
| 22.6 | 3.9 | 16.3 |
| 24.0 | 3.7 | 11.0 |
| 24.2 | 3.7 | 10.2 |
| 24.8 | 3.6 | 15.0 |
| 24.9 | 3.5 | 16.5 |
| 25.4 | 3.5 | 11.9 |
| 25.9 | 3.4 | 56.8 |
| 26.1 | 3.4 | 100 |
| 26.9 | 3.3 | 13.3 |
| 27.4 | 3.2 | 9.6 |
| 30.9 | 2.9 | 10.1 |
| 35.8 | 2.5 | 8.9 |

Crystalline Form V may be characterized by peaks at diffraction angles 2-theta of about 15.9, 21.0, 22.6, 24.0, 26.1, 27.4, and 30.9, ±0.2 degrees. For all analytical data discussed in this application, it should be kept in mind that the exact values obtained can depend on many factors, e.g., the specific instrument, sample preparation, and analyst technique. XRPD peak intensities are particularly influenced by sample preparation and handling techniques. Typical tolerances for 2⊖ peak locations with well-maintained generally available instruments is about 0.2°

An aspect of the present invention provides processes for preparation of crystalline Form V of O-desmethylvenlafaxine succinate, including slurrying any solid form of O-desmethylvenlafaxine succinate in a slurrying solvent for a period of time sufficient to crystallize Form V. Non-limiting examples of suitable slurrying solvents include but are not limited to N,N-dimethylformamide, N,N-dimethylacetamide, and the like.

The suspension in the above slurrying method may be stirred for a period of about 30 minutes to about 30 hours, or longer. Suitable temperatures for the slurrying and stirring may range from about 20° C. to about 60° C., or from about 25° C. to about 35° C. The solid may be isolated by techniques known in the art such as filtration, decantation and the like.

An aspect of the present invention provides a new crystalline form of O-desmethylvenlafaxine succinate, named “Form VI.”

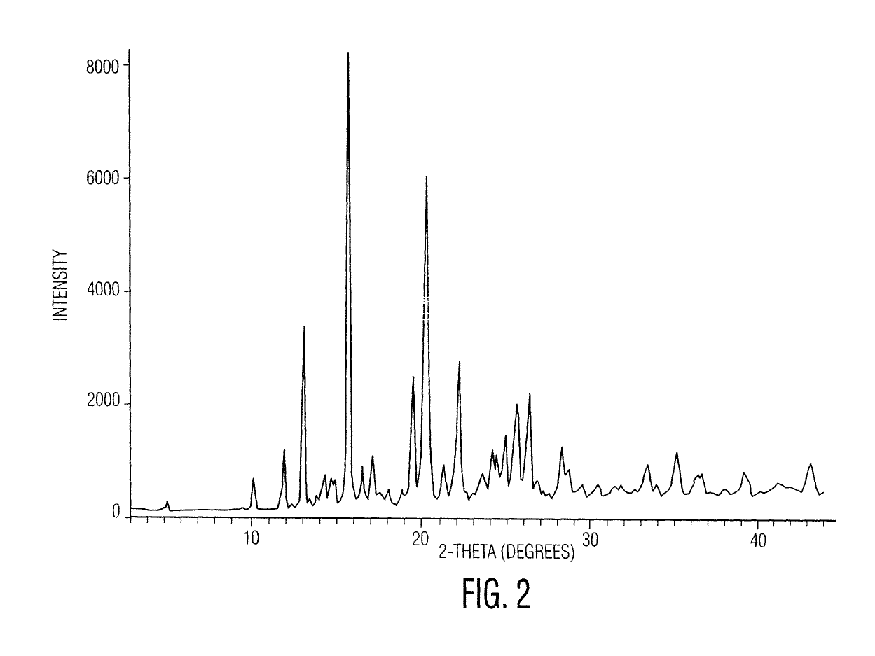

Table 2 contains representative XRPD pattern peak values for crystalline Form VI of O-desmethylvenlafaxine succinate. An example of an XRPD pattern for crystalline Form VI of O-desmethylvenlafaxine succinate is shown in FIG. 2.

TABLE 2

| 2-Theta | | Intensity |
|---|---|---|
| (degrees) | d (Å) | (%) |
| 12.1 | 7.3 | 12.8 |
| 13.2 | 6.7 | 42.6 |
| 15.9 | 5.5 | 100 |
| 16.5 | 5.3 | 10.7 |
| 17.2 | 5.1 | 13.3 |
| 19.6 | 4.5 | 31.3 |
| 20.4 | 4.3 | 79.8 |
| 21.4 | 4.1 | 11.3 |
| 22.4 | 3.9 | 34.3 |
| 24.4 | 3.6 | 15.2 |
| 24.6 | 3.6 | 12.9 |
| 25.2 | 3.5 | 18.3 |
| 25.9 | 3.4 | 26.0 |
| 25.9 | 3.4 | 23.7 |
| 26.6 | 3.3 | 27.7 |
| 28.6 | 3.1 | 15.4 |
| 29.0 | 3.0 | 9.5 |
| 33.8 | 2.6 | 10.5 |
| 35.5 | 2.5 | 14.3 |
| 37.0 | 2.4 | 9.0 |
| 39.5 | 2.2 | 9.5 |
| 43.7 | 2.0 | 12.1 |

Crystalline Form V may be characterized by peaks at diffraction angles 2-theta of about 12.1, 13.2, 15.9, 19.6, 20.4, and 26.7, ±0.2 degrees.

An aspect of the present invention provides processes for preparation of crystalline Form VI of O-desmethylvenlafaxine succinate, including slurrying any solid form of O-desmethylvenlafaxine succinate in a slurrying solvent mixture for a period of time sufficient to crystallize Form VI. Non-limiting examples of a suitable first solvent in the slurring mixture includes but is not limited to dimethylsulfoxide and the like. Non-limiting examples of suitable second solvents in the slurrying mixture include but are not limited to ketones such as methyl isobutyl ketone, methyl ethyl ketone, and mixtures thereof.

The suspension may be stirred for a period of about 30 minutes to about 30 hours, or longer. The volume ratio of the first solvent to the second solvent may generally range from about 1:1 to about 1:3, or about 1:2. Suitable temperatures for the slurrying and stirring range from about 0° C. to about 60° C., or from about 25° C. to about 35° C. The solid may be isolated by techniques known in the art such as filtration, decantation and the like.

Solid forms of O-desmethylvenlafaxine succinate described in the present invention may be formulated into solid pharmaceutical products for oral administration in the form of capsules, tablets, pills, powders or granules. In these compositions, the active ingredient is combined with one or more pharmaceutically acceptable excipients. The drug substance also may be formulated into liquid compositions for oral administration including for example solutions, suspensions, syrups, elixirs and emulsions, containing solvents or vehicles such as water, sorbitol, glycerine, propylene glycol or liquid paraffins.

Compositions for parenteral administration may be suspensions, emulsions or aqueous or non-aqueous, sterile solutions. As a solvent or vehicle, propylene glycol, polyethylene glycol, vegetable oils, especially olive oil, and injectable organic esters, e.g. ethyl oleate, may be employed. These compositions may contain adjuvants, especially wetting, emulsifying and dispersing agents. Sterilization may be carried out in several ways, e.g., using a bacteriological filter, by incorporating sterilizing agents in the composition, by irradiation or by heating. They may be prepared in the form of sterile compositions, which may be dissolved at the time of use in sterile water or any other sterile injectable medium.

Pharmaceutically acceptable excipients include, but are not limited to, diluents such as starch, pregelatinized starch, lactose, powdered cellulose, microcrystalline cellulose, dicalcium phosphate, tricalcium phosphate, mannitol, sorbitol and sugar; binders such as acacia, guar gum, tragacanth, gelatin, polyvinylpyrrolidones, hydroxypropyl celluloses, hydroxypropylmethyl celluloses and pregelatinized starch; disintegrants such as starch, sodium starch glycolate, pregelatinized starch, crospovidones, croscarmellose sodium and colloidal silicon dioxide; lubricants such as stearic acid, magnesium stearate and zinc stearate; glidants such as colloidal silicon dioxide; solubility or wetting enhancers such as anionic or cationic or neutral surfactants, complex forming agents such as various grades of cyclodextrins and resins; release rate controlling agents such as hydroxypropyl celluloses, hydroxymethyl celluloses, hydroxypropyl methylcelluloses, ethyl celluloses, methyl celluloses, various grades of methyl methacrylates, and waxes. Other pharmaceutically acceptable excipients that are of use include but are not limited to film formers, plasticizers, colorants, flavoring agents, sweeteners, viscosity enhancers, preservatives, and antioxidants.

Having described the invention with reference to certain embodiments, other embodiments will become apparent to one skilled in the art from consideration of the specification. Certain specific aspects and embodiments of the invention are further described by the following examples, being provided only for purposes of illustration and not to be construed as limiting the scope of the invention.

### EXAMPLE 1

### PREPARATION OF 1-[CYANO(4-METHOXYPHENYL)METHYL]CYCLOHEXANOL (FORMULA VII)

4-Methoxybenzyl cyanide (100 g) was added to a flask containing sodium methoxide (93 g) and methanol (500 ml) at temperature of about −5° C. to 5° C., over a period of about one hour. Cyclohexanone (87.5 g) was added to the reaction mixture at about −5° C. to 5° C., over a period of about one hour. The reaction mixture was maintained at that temperature for about 4 to about 5 hours until the reaction was complete. The reaction mixture was quenched by the addition of water (1000 ml). The solid was collected by filtration, and was then added to a flask containing ethyl acetate (100 ml) and hexane (1000 ml). The suspension was stirred at about 25° C. for about 3 hours. The solid was separated by filtration, washed with a mixture of ethyl acetate and hexane, and then dried under vacuum. Yield: 150 g.

### EXAMPLE 2

### PREPARATION OF ACETIC ACID SALT OF 1-[2-AMINO-1-(4-METHOXYPHENYL)ETHYL]CYCLOHEXANOL (FORMULA VI)

Glacial acetic acid (300 ml) and 1-[cyano(4-methoxyphenyl)methyl]cyclohexanol (100 g) were placed into an autoclave vessel, into which nickel alloy catalyst (15 g) in glacial acetic acid (300 ml) was added and the vessel was flushed with hydrogen gas two times with a pressure of about 2 kg/cm². The reaction mixture was slowly heated to about 55° C. and it was pressurized with hydrogen gas (up to 17 kg/cm²). The reaction mixture was stirred at about 55° C. under hydrogen pressure (10-15 kg/cm²) for about 4 to about 6 hours. After the completion of the reaction, the mixture was cooled to about 25° C. The catalyst was filtered, the filter was washed with acetic acid and then the acetic acid of the filtrate was distilled completely under vacuum. To the residue, water (500 ml) and toluene (300 ml) were added. The layers were separated. To the aqueous layer, ethyl acetate (500 ml) was added and the mixture was cooled to about 0° C. to about 10° C. Ammonia solution (200 ml, 25%) was added and the mixture was stirred for about 30 minutes at about 25° C. The organic layer was separated. The aqueous layer was extracted again with ethyl acetate (2×200 ml). The organic layers were combined. After the solvent was distilled under vacuum, the residue was taken in ethyl acetate (400 ml) and glacial acetic acid (35 ml) was added slowly at about 25° C. The mixture was heated to reflux (about 77° C.) for about 30 minutes. The mixture was cooled to about 0° C. and solid was filtered, washed with ethyl acetate and dried. Yield: 88 g.

### EXAMPLE 3

### PREPARATION OF 1-[2-(DIMETHYLAMINO)-1-(4-METHOXY-PHENYL)ETHYL]CYCLOHEXANOL HYDROCHLORIDE (FORMULA II)

A mixture of 1-[2-amino-1-(4-methoxyphenyl)ethyl]cyclohexanol (50 g), formaldehyde (40%, 73 ml), formic acid (22.3 g) and water (250 ml) was heated at about 90° C. to about 100° C. for about 20 to 22 hours. The reaction mass was cooled to about 25° C. and washed with dichloromethane (3×50 ml). The aqueous layer was then cooled to about 0° C. and the pH of the solution was adjusted to about 9 to 10 by aqueous sodium hydroxide solution (13 g of sodium hydroxide solution in 250 ml of water). The product was extracted with toluene (2×50 ml). The organic layers were combined and the pH was adjusted to about 3 to 5 by hydrogen chloride in isopropanol (12%, 50 ml). The mixture was cooled to about 0° C. and stirred for about 30 minutes. The precipitated solid was filtered and the solid was washed with toluene (50 ml). The obtained solid was then mixed with isopropanol (300 ml) and heated to reflux for about 30 minutes. The mixture was cooled to about 0° C. The precipitated solid was filtered, washed with isopropanol (50 ml) and dried. Yield: 41 g.

### EXAMPLE 4

### PREPARATION OF 1-[2-(DIMETHYLAMINO)-1-(4-METHOXY-PHENYL)ETHYL]CYCLOHEXANOL HYDROCHLORIDE (FORMULA II)

A stirred mixture of 1-[2-amino-1-(4-methoxy phenyl)ethyl]cyclohexanol acetate (55.0 g), formic acid (25 ml), 40% formaldehyde solution (92 ml) and water (275 ml) was heated at about 95° C. for about 19 hours. The reaction mass was cooled and washed with chloroform (4×55 ml). The washings were discarded. The aqueous layer was then cooled to 5° C. and 48% sodium hydroxide solution (25 ml) was added to it. The product was extracted from the alkaline aqueous layer with chloroform (3×100 ml). The organic layer was then evaporated under reduced pressure to yield an oily residue, which was dissolved in isopropyl alcohol (225 ml). The resultant solution was acidified with isopropyl alcoholic hydrogen chloride until a pH of about 2 was achieved. The precipitated solid was filtered and washed with isopropyl alcohol (25 ml). It was then dried at about 60° C. to yield the desired compound of Formula II. Yield: 44.0 g.

### EXAMPLE 5

### PREPARATION OF 1-[2-DIMETHYLAMINE (4-HYDROXYPHENYL) ETHYL] CYCLOHEXANOL (FORMULA I) USING SODIUM HYDROXIDE AND DIMETHYLSULFORXIDE

Sodium hydroxide (11.5 g) and water (10 ml) were placed into a round bottom flask equipped with a Dean-Stark apparatus and stirred for about 5 minutes. A solution of dodecanethiol (48.4 g) in toluene (250 ml) was added and the mixture was heated to about 110° C. for about 1-2 hours to remove water azotropically. After water removal, toluene was completely distilled off under vacuum to afford the sodium salt of dodecanethiol.

Venlafaxine hydrochloride (25 g), dichloromethane (75 ml) and water (50 ml) were placed into a round bottom flask and stirred for about 5-10 minutes at about 0° C.-10° C. The pH of the reaction solution was adjusted to about 10 by adding 5% sodium hydroxide solution (65 ml). The obtained reaction solution was then warmed to about 25° C.-30° C. The layers were separated and the aqueous layer was washed with dichloromethane (25 ml). The combined organic layer was dried over sodium sulphate. The organic solvent was completely distilled under vacuum to afford venlafaxine free base.

The above obtained sodium salt of dodecanethiol was charged into a clean and dry round bottom flask. Venlafaxine free base dissolved in dimethylsulfoxide (100 ml) was added slowly through a dropper over a 20 to 30-minute period at about 25° C.-30° C. and stirred for about 5-10 minutes. The reaction mass was heated to about 180° C.-190° C. and maintained for 1-2 hours or until the completion of the reaction. The reaction mass was cooled to about 25° C.-30° C., quenched by the addition of water (500 ml) and stirred for about 10-15 minutes. The solution was then cooled to about 0° C.-10° C. The pH of the solution was adjusted to about 3-4 by conc. HCl (29 ml) and stirred for about 10-15 minutes. The pH of the reaction solution was then adjusted to about 10 by addition of aqueous sodium hydroxide solution (33 ml). The resultant reaction solution was stirred for about 45-60 minutes at about 0° C.-10° C. for solid formation. The formed solid was filtered, washed with water (200 ml) and suction dried for about 10-15 minutes. The obtained cake was further washed with cyclohexane (100 ml) and then dried under a vacuum at about 60° C.-70° C. for about 3-4 hours to afford 14.8 g of the title compound. HPLC purity: 95.26%.

### EXAMPLE 6

### ALTERNATE PREPARATION OF 1-[2-DIMETHYLAMINE (4-HYDROXYPHENYL) ETHYL]CYCLOHEXANOL (FORMULA I) USING POTASSIUM HYDROXIDE AND DIMETHYLSULFOXIDE

Potassium hydroxide (16 g) and water (15 ml) were placed into a round bottom flask equipped with a Dean-Stark apparatus and stirred for about 5 minutes. A solution of dodecanethiol (48.4 g) in toluene (250 ml) was added and the mixture was heated to about 110° C. for about 1-2 hours to remove water azotropically. After water removal, toluene was completely distilled off under vacuum to afford the sodium salt of dodecanethiol.

Venlafaxine hydrochloride (25 g), dichloromethane (75 ml) and water (50 ml) were placed into a round bottom flask and stirred for about 5-10 minutes. The solution was then cooled to about 0° C.-10° C. The pH of the solution was adjusted to about 10 by addition of 5% sodium hydroxide solution (65 ml). The solution was then warmed to about 25° C.-30° C. The organic and aqueous layers were separated and the aqueous layer was washed with dichloromethane (25 ml). The combined organic layer was dried over sodium sulphate. The organic solvent was then distilled off completely under vacuum to afford venlafaxine free base.

The above-obtained potassium salt of dodecanethiol was charged into a clean dry round bottom flask. Venlafaxine free base dissolved in dimethylsulfoxide (100 ml) was slowly added through a dropper over a 20 to 30-minute period at about 25° C.-30° C. and stirred for about 5-10 minutes. The mass was heated to about 160° C.-180° C. and maintained for about 1-2 hours or until the completion of the reaction. The reaction mass was cooled to about 25° C.-30° C. and quenched by the addition of water (500 ml) and stirred for about 10-15 minutes. The solution was then further cooled to about 0° C.-10° C. The pH of the reaction solution was adjusted to about 3-4 by conc. HCl (34 ml) and stirred for about 10-15 minutes. The pH of the reaction solution was then adjusted to about 10 by addition of aqueous sodium hydroxide solution (38 ml). The resultant solution was stirred for about 45-60 minutes at about 0° C.-10° C. for solid formation. The formed solid was filtered, washed with water (200 ml) and suction dried for about 10-15 minutes. The obtained cake was further washed with cyclohexane (100 ml) and then dried under a vacuum at about 60° C.-70° C. for about 3-4 hours to afford 13.4 g of the title compound. HPLC purity: 95.02%.

### EXAMPLE 7

### ALTERNATE PREPARATION OF 1-[2-DIMETHYLAMINE (4-HYDROXYPHENYL) ETHYL]CYCLOHEXANOL (FORMULA I) USING POTASSIUM HYDROXIDE AND N-METHYLPYRROLIDONE

Potassium hydroxide (16 g) and water (15 ml) were placed into a round bottom flask equipped with a Dean-Stark apparatus and stirred for about 5 minutes. A solution of dodecanethiol (48.4 g) in toluene (200 ml) was added and the mixture heated to about 110° C. for about 1-2 hours to remove water azotropically. After removal of the water, toluene was distilled off completely under vacuum to afford the sodium salt of dodecanethiol.

Venlafaxine hydrochloride (25 g), dichloromethane (75 ml) and water (50 ml) were placed into a round bottom flask and stirred for about 5-10 minutes. The solution was then cooled to about 0° C.-10° C. The pH of the reaction solution was adjusted to about 10 by addition of 5% sodium hydroxide solution (65 ml). The solution was then warmed to about 25° C.-30° C. The organic and aqueous layers were separated and the aqueous layer was washed with dichloromethane (25 ml). The combined organic layer was dried over sodium sulphate. The solvent was completely distilled off under vacuum to afford venlafaxine free base.

The above-obtained potassium salt of dodecanethiol was charged into a clean dry round bottom flask. Venlafaxine free base dissolved in N-methyl pyrrolidone (100 ml) was added slowly through a dropper over 20 to 30 minutes at about 25° C.-30° C. and stirred for about 5-10 minutes. The mass was heated to about 180° C.-190° C. and maintained for about 1-2 hours or until the completion of the reaction. The reaction mass was cooled to about 25° C.-30° C., quenched by adding water (500 ml) and stirred for about 10-15 minutes. The solution was cooled to about 0° C.-10° C. The pH of the solution was adjusted to about 3-4 by conc. HCl (25 ml) and stirred for about 10-15 minutes. The pH of the reaction solution was then adjusted to about 10 by addition of aqueous sodium hydroxide solution (15 ml). The resultant solution was stirred for about 45-60 minutes at about 0° C.-10° C. for solid formation. The formed solid was filtered, washed with water (200 ml) and suction dried for about 10-15 minutes. The obtained cake was further washed with cyclohexane (100 ml) and then dried under a vacuum at about 60° C.-70° C. for about 3-4 hours to afford 20.1 g of the title compound. HPLC purity: 99.55%.

### EXAMPLE 8

### ALTERNATE PREPARATION OF 1-[2-DIMETHYLAMINE (4-HYDROXYPHENYL) ETHYL]CYCLOHEXANOL (FORMULA I) USING SODIUM HYDROXIDE AND N-METHYL PYRROLIDONE

Sodium hydroxide (10.1 g) and water (13 ml) were placed into a round bottom flask equipped with a Dean-Stark apparatus and stirred for about 5 minutes. A solution of dodecanethiol (42.6 g) in toluene (220 ml) was added and the mixture heated to about 110° C. for about 1-2 hours to remove water azotropically. After removal of water, the toluene was completely distilled off under vacuum to afford the sodium salt of dodecanethiol.

Venlafaxine hydrochloride (22.0 g), dichloromethane (65 ml) and water (45 ml) were placed into a round bottom flask and stirred for about 5-10 minutes. The solution was then cooled to about 0° C.-10° C. The pH of the solution was adjusted to about 10 by addition of 5% sodium hydroxide solution (57 ml). The solution was then warmed to about 25° C.-30° C. The organic and aqueous layers were separated and the aqueous layer was washed with dichloromethane (25 ml). The combined organic layer was dried over sodium sulphate. The organic solvent was completely distilled off under vacuum to afford venlafaxine free base.

The above-obtained sodium salt of dodecanethiol was charged into a clean dry round bottom flask. Venlafaxine free base dissolved in N-methylpyrrolidone (90 ml) was added slowly through a dropper over 20 to 30-minutes at about 25° C.-30° C. and stirred for about 5-10 minutes. The mass was heated to about 180° C.-190° C. and maintained for about 1-2 hours or until the completion of the reaction. The reaction mass was cooled to about 25° C.-30° C., quenched by adding water (450 ml) and stirred for about 10-15 minutes. The solution was then cooled to about 0° C.-10° C. The pH of the solution was adjusted to about 3-4 by conc. HCl (20 ml) and stirred for about 10-15 minutes. The pH of the solution was again adjusted to about 10 by addition of aqueous sodium hydroxide solution (8 ml). The resultant solution was stirred for about 45-60 minutes at about 10° C.-30° C. for solid formation. The formed solid was filtered, washed with water (300 ml) and suction dried for about 10-15 minutes. The obtained cake was further washed with cyclohexane (100 ml) and then dried under a vacuum at about 60° C.-70° C. for about 3-4 hours to afford 14.8 g of the title compound. HPLC purity: 99.76%.

### EXAMPLE 9

### PURIFICATION OF 1-[2-DIMETHYLAMINE (4-HYDROXYPHENYL) ETHYL]CYCLOHEXANOL (FORMULA I)

O-desmethylvenlafaxine (20 g) was charged into a round bottom flask containing isopropanol (500 ml) and stirred for about 5-10 minutes. The mixture was heated to reflux to get a clear solution. Carbon black (4 g) was added and stirred for about 10-15 minutes. The reaction solution was then filtered through a Hyflow bed. The obtained clear solution was charged into a round bottom flask, cooled to about 0° C.-5° C., and stirred for about 1-2 hours for solid formation. The formed solid was filtered, washed with isopropyl alcohol and suction dried. The obtained solid was dried at about 60° C.-70° C. under vacuum to afford 14.7 g of the title compound. HPLC purity: 99.86%.

### EXAMPLE 10

### PREPARATION OF O-DESMETHYLVENLAFAXINE SUCCINATE

n-Butanol (50 ml), O-desmethylvenlafaxine (5 g), succinic acid (2.3 g), and water (5 ml) were charged into a round bottom flask with stirring. The mixture was heated to a temperature of about 70° C. to about 80° C. The solution was stirred for about 20 to about 30 minutes at a temperature of about 75° C. to about 80° C. and then cooled to a temperature of about 0° C. to about 5° C. for solid formation. The suspension was stirred for about 20 to about 30 minutes and then filtered. The solid was washed with chilled n-butanol (5 ml) and dried for about 5 hours at a temperature of about 55° C. to about 60° C., to afford 7.0 g of title compound. Purity: 99.91% by HPLC.

### EXAMPLE 11

### PREPARATION OF O-DESMETHYLVENLAFAXINE SUCCINATE

Isopropyl alcohol (150 ml) and O-desmethylvenlafaxine (5 g) were charged into a round bottom flask with stirring. The mixture was heated to reflux temperature. A solution of succinic acid (2.3 g of succinic acid in 10 ml of water) was added to the solution at a temperature of about 80° C. to about 85° C. and then the mixture was cooled to a temperature of about 25° C. to about 35° C. for solid formation. The suspension was stirred for about 1 to about 2 hours and then filtered. The solid was washed with chilled isopropyl alcohol (10 ml) and dried for about 2 to about 3 hours at a temperature of about 55° C. to about 60° C. to afford 6.4 g of title compound.

### EXAMPLE 12

### PREPARATION OF O-DESMETHYLVENLAFAXINE SUCCINATE

Methyl isobutyl ketone (250 ml) and O-desmethylvenlafaxine (5 g) were charged into a round bottom flask with stirring. The mixture was heated to a temperature of about 80° C. to about 90° C. and stirred for about 30 to about 45 minutes. A solution of succinic acid (2.3 g of succinic acid in 10 ml of water) was added to the solution over a period of about 30 to about 45 minutes at a temperature of about 80° C. to about 90° C. and then cooled to a temperature of about 25° C. to about 35° C. for solid formation. The suspension was stirred for about 1 hour and then filtered. The solid was washed with methyl isobutyl ketone (10 ml) and dried for about 2 to about 3 hours at a temperature of about 55° C. to about 60° C. to afford 6.8 g of title compound.

### EXAMPLE 13

### PREPARATION OF O-DESMETHYLVENLAFAXINE SUCCINATE

Methyl t-butyl ether (50 ml), O-desmethylvenlafaxine (5 g), succinic acid (2.3 g), and water (5 ml) were charged into a round bottom flask. The mixture was heated to reflux temperature and then cooled to a temperature of about 25° C. to about 35° C. followed by stirring for about 1 hour for solid formation. The suspension was filtered and washed with methyl t-butyl ether (5 ml). The obtained solid was dried for about 2 to about 3 hours at a temperature of about 55° C. to about 60° C. to afford 7.2 g of title compound. Purity: 99.97%.

### EXAMPLE 14

### PREPARATION OF O-DESMETHYLVENLAFAXINE SUCCINATE

Ethyl acetate (50 ml), O-desmethylvenlafaxine (5 g), succinic acid (2.3 g), and water (5 ml) were charged into a round bottom flask. The mixture was heated to reflux temperature and stirred for about 30 minutes. The solution was cooled to a temperature of about 25° C. to about 35° C. followed by stirring for about 1 to about 2 hours for solid formation. The suspension was filtered and washed with ethyl acetate (5 ml). The obtained solid was dried for about 2 to about 3 hours at a temperature of about 55° C. to about 60° C. to afford 7.2 g of title compound. Purity: 99.98% by HPLC.

### EXAMPLE 15

### PREPARATION OF CRYSTALLINE FORM V

O-desmethylvenlafaxine succinate (800 mg) was suspended in N,N-dimethylformamide (2 ml) at a temperature of about 30° C. and stirred for about 24 hours. The resultant suspension was filtered and suction dried under vacuum to afford 420 mg of title crystalline form (XRPD pattern is shown in FIG. 1).

### EXAMPLE 16

### PREPARATION OF CRYSTALLINE FORM VI

O-desmethylvenlafaxine succinate (2 g) was suspended in dimethylsulfoxide (3 ml) at a temperature of about 30° C. and stirred for about 5 minutes. Methyl isobutyl ketone (6 ml) was added to the suspension and stirred for about 24 hours at a temperature of about 30° C. The resulting suspension was filtered and the solid dried for about 7 hours at a temperature of about 50° C. to afford 1.1 g of title crystalline form (XRPD pattern is shown in FIG. 2).

### EXAMPLE 17

### PREPARATION OF STABLE AMORPHOUS O-DESMETHYLVENLAFAXINE SUCCINATE SOLID DISPERSION WITH POVIDONE

Povidone (3.0 g), O-desmethylvenlafaxine succinate (3.0 g) and methanol (75 ml) were charged into a clean and dry round bottom flask. The suspension was heated to a temperature of about 50° C. and the solution was filtered. The obtained filtrate was concentrated completely at a temperature of about 50° C. and the solid was dried for about 1 hour under vacuum, to afford 5 g of O-desmethylvenlafaxine succinate amorphous solid dispersion with povidone. It is characterized by XRPD pattern substantially as illustrated by FIG. 3. The obtained solid was packaged in two self-sealing polyethylene bags.

One bag was stored for 1 month at room temperature under normal atmospheric conditions and then checked for polymorphic stability. The material was found to retain its polymorphic form after one month of storage, as indicated by its XRPD pattern.

The second bag was stored for 1 month at a temperature of about 0° C. to about 5° C. and then checked for polymorphic stability. The material was found to retain its polymorphic form after one month of storage, as indicated by its XRPD pattern.

### EXAMPLE 18

### PREPARATION OF STABLE AMORPHOUS O-DESMETHYLVENLAFAXINE SUCCINATE SOLID DISPERSION WITH POVIDONE

O-desmethylvenlafaxine (20.0 g) and methanol (240 ml) were charged into a clean and dry round bottom flask and stirred for about 5 to 15 minutes at about 30° C. Succinic acid (9.24 g) was added to the solution and stirred for about 1 hour at about 30° C. Povidone (39.24 g), pretreated with sodium metabisulfite, in methanol was added to the solution and stirred for about 30 minutes at about 30° C. The mixture was filtered and the filer was washed with methanol (40 ml). The obtained filtrate was concentrated completely at a temperature of about 80° C. and the solid residue was dried for about 5 hours under vacuum to afford 36.9 g of O-desmethylvenlafaxine succinate amorphous solid dispersion with povidone. It is characterized by an XRPD pattern substantially as shown in FIG. 3.

The obtained solid was packaged in two self-sealing polyethylene bags. One bag was stored for 1 month at room temperature under normal atmospheric conditions and checked for polymorphic stability. The material was found to retain its polymorphic form after one month of storage, as indicated by its XRPD pattern.

The second bag was stored for 1 month at a temperature of about 0° C. to about 5° C. and checked for polymorphic stability. The material was found to retain its polymorphic form after one month of storage, as indicated by its XRPD pattern.

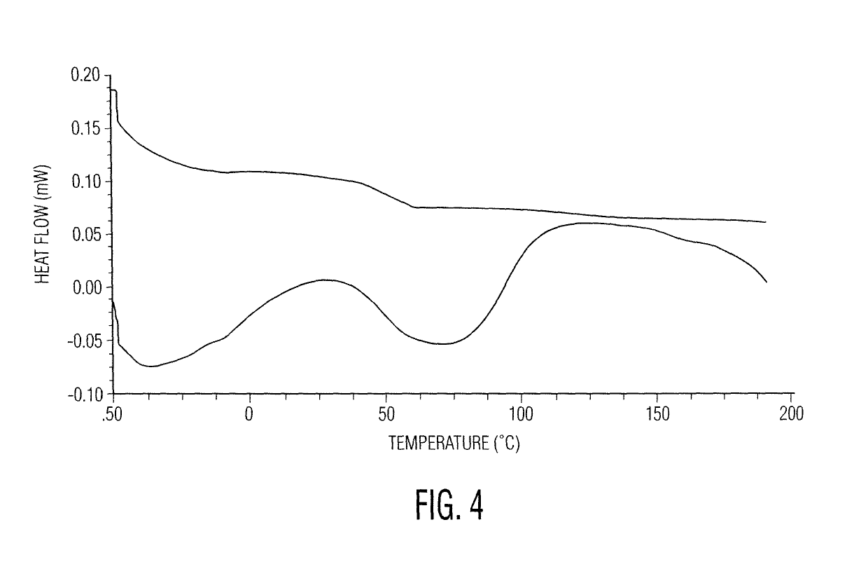

A typical DSC curve for the amorphous solid dispersion O-desmethylvenlafaxine succinate in combination with povidone is shown in FIG. 4.

### EXAMPLE 19

### PREPARATION OF STABLE AMORPHOUS O-DESMETHYLVENLAFAXINE SUCCINATE SOLID DISPERSION WITH HPMC

Hydroxypropyl methylcellulose (HPMC; 3.0 g), O-desmethylvenlafaxine succinate (3.0 g) and methanol (130 ml) were charged into a round bottom flask. The mixture was heated to a temperature of about 50° C. and stirred for about 5 minutes. The solution was filtered and then the obtained filtrate was concentrated completely at a temperature of about 50° C. under vacuum. The residue was dried at a temperature of about 50° C. for one hour to afford 5.5 g of title compound. It is characterized by the XRPD pattern substantially as shown in FIG. 3.

The obtained solid was packaged in a self-sealing polyethylene bag. The bag was stored for 1 month at a temperature of about 0° C. to about 5° C. and checked for polymorphic stability. The material was found to retain its polymorphic form after one month of storage, as indicated by its XRPD pattern.

### EXAMPLE 20

### PREPARATION OF STABLE AMORPHOUS O-DESMETHYLVENLAFAXINE SUCCINATE SOLID DISPERSION WITH ETHYLCELLULOSE

Ethyl cellulose (3.0 g), O-desmethylvenlafaxine succinate (3.0 g) and methanol (150 ml) were charged into a round bottom flask. The suspension was heated to a temperature of about 50° C. and the obtained solution was filtered. The filtrate was distilled completely under vacuum at a temperature of about 50° C. and the solid was dried for about 1 hour at a temperature of about 50° C. to afford 5.5 g of title compound. It is characterized by XRPD pattern substantially as shown in FIG. 3.

The obtained solid was packaged in a self-sealing polyethylene bag. The bag was stored for 1 month at a temperature of about 0° C. to about 5° C. and checked for polymorphic stability. The material was found to retain its polymorphic form after one month of storage, as indicated by its XRPD pattern.

### EXAMPLE 21

### PREPARATION OF STABLE AMORPHOUS O-DESMETHYLVENLAFAXINE SUCCINATE SOLID DISPERSION WITH POLYETHELENE GLYCOL

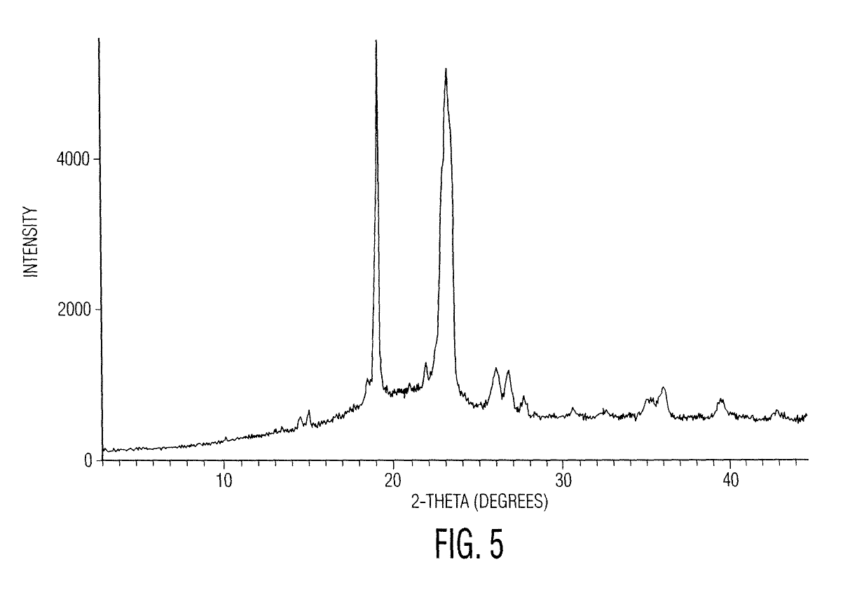

O-desmethylvenlafaxine succinate (3.0 g) and polyethylene glycol 6000 (3.0 g) were dissolved in methanol (75 ml) at a temperature of about 50° C. and the solution was filtered. The obtained filtrate was concentrated completely under vacuum at a temperature of about 50° C. and the solid was subjected for drying for about 1 hour at a temperature of about 50° C. to afford 5.5 g of title compound. It is characterized by an XRPD pattern substantially as shown in FIG. 5. The well defined peaks present in the XRPD pattern are attributed to polyethylene glycol 6000, which is crystalline, and the O-desmethylvenlafaxine succinate that is present in the resultant solid dispersion is observed to be amorphous.

The obtained solid was packaged in a self-sealing polyethylene bag. The bag was stored for 1 month at a temperature of about 0° C. to about 5° C. and checked for polymorphic stability. The material was found to retain its polymorphic form after one month of storage, as indicated by maintenance of the original XRPD pattern.

1. An amorphous solid dispersion comprising O-desmethylvenlafaxine succinate and at least one pharmaceutically acceptable carrier.

2. The amorphous solid dispersion of claim 1, wherein a pharmaceutically acceptable carrier comprises one or more of a povidone, gum, ethylcellulose, hydroxypropyl methylcellulose, microcrystalline cellulose, cyclodextrin, gelatin, hypromellose phthalate, sugar, polyhydric alcohol, polyethylene glycol, polyethylene oxide, polyoxyalkylene derivative, methacrylic acid copolymer, polyvinyl alcohol, or propylene glycol derivative.

3. The amorphous solid dispersion of claim 1, wherein a pharmaceutically acceptable carrier comprises a povidone, hydroxypropylmethyl cellulose, ethyl cellulose, or polyethylene glycol.

4. The amorphous solid dispersion of claim 1, having an X-ray powder diffraction pattern substantially according to the pattern of FIG. 3.

5. A process for preparing an amorphous solid dispersion comprising O-desmethylvenlafaxine succinate and at least one pharmaceutically acceptable carrier of claim 1, which includes:
a) providing:
i) a solution or mixture of O-desmethylvenlafaxine succinate and at least one pharmaceutically acceptable carrier in a solvent; or
ii) a mixture or a solution of O-desmethylvenlafaxine, succinic acid, and at least one pharmaceutically acceptable carrier in a solvent;
b) isolating a solid dispersion; and
c) optionally, drying the solid dispersion.

6. The process of claim 5, wherein a pharmaceutically acceptable carrier comprises one or more of a povidone, gum, ethylcellulose, hydroxypropyl methyl cellulose, microcrystalline cellulose, cyclodextrin, gelatin, hypromellose phthalate, sugar, polyhydric alcohol, polyethylene glycol, polyethylene oxide, polyoxyalkylene derivative, methacrylic acid copolymer, polyvinyl alcohol, or propylene glycol derivative.

7. The process of claim 5, wherein a pharmaceutically acceptable carrier comprises a povidone, hydroxypropyl methylcellulose, ethyl cellulose, or polyethylene glycol.

8. The process of claim 5, wherein a pharmaceutically acceptable carrier is optionally pretreated to remove contaminants, before a dispersion is formed.

9. Crystalline O-desmethylvenlafaxine succinate Form V.

10. Crystalline O-desmethylvenlafaxine succinate Form V of claim 9, characterized by an powder X-ray powder diffraction pattern with copper Kα radiation having peaks at about 15.9, 21.0, 22.6, 24.0, 26.1, 27.4, and 30.9, ±0.2 degrees two-theta.

11. Crystalline O-desmethylvenlafaxine succinate Form V of claim 9, having an X-ray powder diffraction pattern with copper Kα radiation substantially in accordance with the pattern of FIG. 1.

12. A process for preparing crystalline O-desmethylvenlafaxine succinate Form V of claim 11, comprising:
a) providing a suspension of O-desmethylvenlafaxine succinate in N,N-dimethylformamide or N,N-dimethylacetamide; and
b) stirring the suspension for a time sufficient to form crystalline O-desmethylvenlafaxine succinate Form V.

13. The process of claim 12, wherein the solvent is N,N-dimethylformamide.

14. Crystalline O-desmethylvenlafaxine succinate Form VI.

15. Crystalline O-desmethylvenlafaxine succinate Form VI of claim 14, characterized by an X-ray powder diffraction pattern with copper Kα radiation having peaks at about 12.1, 13.2, 15.9, 19.6, 20.4, and 26.7, ±0.2 degrees two-theta.

16. Crystalline O-desmethylvenlafaxine succinate Form VI of claim 14, having an X-ray powder diffraction pattern substantially in accordance with the pattern of FIG. 2.

17. A process for preparing crystalline O-desmethylvenlafaxine succinate Form VI of claim 14 comprising steps of:
a) providing a suspension of O-desmethylvenlafaxine succinate in an organic solvent mixture comprising at least two of dimethylsulfoxide, methyl isobutyl ketone, and methyl ethyl ketone; and
b) stirring the mixture for a time sufficient to form crystalline O-desmethylvenlafaxine succinate Form VI.

18. The process of claim 17, wherein the organic solvent is a mixture of dimethylsulfoxide and methyl isobutyl ketone.

19. A process for preparing O-desmethylvenlafaxine or an acid addition salt thereof, comprising:
a) reducing 1-[cyano(4-methoxyphenyl)methyl]cyclohexanol in the presence of a catalyst to form 1-[2-amino-1-(4-methoxyphenyl)ethyl]cyclohexanol;
b) optionally converting 1-[2-amino-1-(4-methoxyphenyl)ethyl]cyclohexanol to an acid addition salt;
c) methylation of 1-[2-amino-1-(4-methoxyphenyl)ethyl]cyclohexanol or an acid addition salt thereof to obtain venlafaxine, and optionally converting venlafaxine into an acid addition salt;
d) demethylating venlafaxine or an acid addition salt thereof with a metal salt of dodecanethiol in an organic solvent;
e) optionally, crystallizing O-desmethylvenlafaxine from an organic solvent; and
f) optionally, converting O-desmethylvenlafaxine into a pharmaceutically acceptable salt.

20. The process of claim 19, wherein the catalyst comprises an activated nickel catalyst.

21. The process of claim 19, wherein an acid in step b) comprises acetic acid or hydrochloric acid.

22. The process of claim 19, wherein a solvent for step d) comprises N,N-dimethylformamide, dimethylsulfoxide, N,N-dimethylacetamide, N-methylpyrrolidone, hexamethylphosphoramide, methyl cellosolve, or a mixture of two or more thereof.

23. The process of claim 19, wherein a solvent for step e) comprises acetone, methyl ethyl ketone, butanone, ethanol, methanol, isopropanol, tetrahydrofuran, 1,4-dioxane, ethyl acetate, propyl acetate, t-butyl acetate, water, or a mixture of two or more thereof.

24. A process for preparing substantially pure O-desmethylvenlafaxine or a pharmaceutically acceptable salt thereof, comprising:
a) reacting dodecanethiol with a suitable base in a solvent to afford a metal salt of dodecanethiol;
b) reacting venlafaxine or its acid addition salt with a metal salt of dodecanethiol in a solvent under suitable reaction conditions;
c) optionally, crystallizing O-desmethylvenlafaxine from a solvent; and
d) optionally, converting O-desmethylvenlafaxine into a pharmaceutically acceptable salt.

25. The process of claim 24, wherein a base for step a) comprises an alkali metal hydroxide, alkali metal carbonate, or alkali metal bicarbonate.

26. The process of claim 24, wherein a solvent for step a) comprises toluene, xylene, n-hexane, n-heptane, cyclohexane, or a mixture of two or more thereof.

27. The process of claim 24, wherein a solvent for step b) comprises N,N-dimethylformamide, dimethylsulfoxide, N,N-dimethylacetamide, N-methylpyrrolidone, hexamethylphosphoramide, methyl cellosolve, or a mixture of two or more thereof.

28. The process of claim 24, wherein a solvent for step c) comprises acetone, ethyl methyl ketone, butanone, ethanol, methanol, isopropanol, tetrahydrofuran, 1,4-dioxane, ethyl acetate, propyl acetate, t-butyl acetate, water, or a mixture of two or more thereof.

29. A process for preparing O-desmethylvenlafaxine succinate comprising:
a) providing a mixture of O-desmethylvenlafaxine and succinic acid in a solvent;
b) heating the mixture; and
c) cooling to form crystals of O-desmethylvenlafaxine succinate.

30. The process of claim 29, wherein a solvent comprises methanol, ethanol, isopropanol, 1,4-dioxane, diethyl ether, tetrahydrofuran, diisopropyl ether, methyl tertiary-butyl ether, toluene, xylene, n-hexane, n-heptane, cyclohexane, ethyl acetate, n-propyl acetate, n-butyl acetate, tertiary-butyl acetate, acetonitrile, propionitrile, dichloromethane, ethylene dichloride, chloroform, a mixture of two or more thereof, or a combination of a solvent or mixture with water.

31. A process for preparation of a compound having Formula IV, comprising: 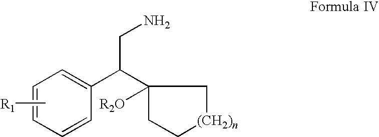
catalytic hydrogenation of phenylacetonitrile of Formula V, 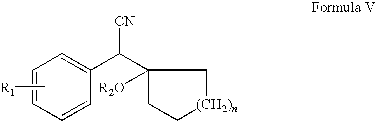
wherein: R₁ is H, —OH, amino, alkylamino, alkylamido, halo, unsubstituted or substituted alkyl or alkoxy; R₂ is hydrogen or a hydroxy protecting group; and n is 1, 2 or 3; in the presence of a catalyst.

32. The process of claim 31, wherein a catalyst comprises an activated nickel catalyst.

33. The process of claim 31, wherein an acid comprises acetic acid or hydrochloric acid.
